(19) United States
(12) Patent Application Publication
(10) Pub. No.: US 2025/0140407 A1
(43) Pub. Date: May 1, 2025

(54) SYSTEMS AND METHODS FOR IDENTIFYING AND TREATING PRIMARY AND LATENT INFECTIONS AND/OR DETERMINING TIME SINCE INFECTION

(71) Applicants: Andrew HEDERMAN, Barnstead, NH (US); Margaret E. ACKERMAN, Hanover, NH (US); Arnaud MARCHANT, Braine l'Alleud (BE); Daniele LILLERI, Pavia (IT); TRUSTEES OF DARTMOUTH COLLEGE, Hanover, NH (US); UNIVERSITÉ LIBRE DE BRUXELLES, Brussels (BE); FONDAZIONE IRCCS POLICLINICO SAN MATTEO, Pavia (IT)

(72) Inventors: Andrew HEDERMAN, Barnstead, NH (US); Margaret E. ACKERMAN, Hanover, NH (US); Arnaud MARCHANT, Braine l'Alleud (BE); Daniele LILLERI, Pavia (IT)

(21) Appl. No.: 18/837,293

(22) PCT Filed: Feb. 10, 2023
(86) PCT No.: PCT/US2023/062376

§ 371 Date: Aug. 9, 2024

Related U.S. Application Data
(60) Provisional application No. 63/418,288, filed on Oct. 21, 2022.
(60) Provisional application No. 63/309,233, filed on Feb. 11, 2022.

(51) Int. Cl. G16H 50/20; G16B 15/00; G16H 20/10; G16H 50/70

(52) U.S. Cl. CPC G16H 50/20; G16B 15/00; G16H 20/10; G16H 50/70

(57) ABSTRACT

Systems and methods for identifying subjects as having latent or primary herpesvirus infections and/or determining the time since the subject was exposed to or infected with a herpesvirus using machine learning algorithms are disclosed. An example method includes detecting in a bodily fluid sample from a subject a set of anti-virus antibody features and generating an input vector that includes data indicative of the anti-virus antibody features of the subject. The method also includes applying the input vector to a trained machine learning algorithm that is configured to generate an assigned classification to the subject. The assigned classification is one of a plurality of potential classifications of the machine learning algorithm. The method also includes determining whether the subject is a suitable candidate for therapeutic intervention based on the assigned classification and, responsive to determining that the subject is suitable, providing the therapeutic intervention to the subject to improve health outcomes.

### CROSS-REFERENCE TO RELATED APPLICATIONS

This application claims the benefit of U.S. Provisional Patent Application No. 63/309,233, which was filed on Feb. 11, 2022 and U.S. Provisional Patent Application No. 63/418,288, which was filed on Oct. 21, 2022, each of which are incorporated by reference herein in their entirety.

### BACKGROUND

The presence of a virus in a host can be detected by analyzing a sample taken from a host to identify genetic material and/or to identify proteins related to the virus. The transmission of a virus from host to host can depend on a number of factors. For example, the mode of transmission, such as via bodily fluids or airborne, can impact the likelihood of a virus not being transmitted from one host to another. Additionally, genomic information of the host and genomic information of the virus can have an influence on the transmission of the virus.

Human cytomegalovirus (HCMV) is an enveloped, double-stranded DNA virus of the herpesvirus family, which includes herpes simplex virus types 1 and 2, varicella-zoster virus, and Epstein-Barr virus. The virion is composed of the double-stranded, 235-kb DNA genome enclosed in an icosahedral protein capsid, which itself is surrounded by a proteinaceous layer termed the tegument and, finally, a lipid envelope. The surface of the virion is decorated by several glycoprotein complexes that mediate viral entry and membrane fusion.

Certain groups are at high risk for serious complications from CMV infection, including infants infected in utero (congenital CMV infection) and individuals with compromised immune systems, such as organ transplant recipients and patients with AIDS.

Serologic tests that detect anti-CMV antibodies (IgM and IgG) are widely available from commercial laboratories. The enzyme-linked immunosorbent assay (ELISA) is the most common serologic test for measuring antibody to CMV. Following primary CMV infection (i.e., infection in a previously seronegative individual), IgG antibodies have low binding strength (low avidity) then over 2-4 months mature to high binding strength (high avidity). A positive test for anti-CMV IgG indicates that a person was infected with CMV at some time during their life but does not indicate when a person was infected. Positive CMV IgM indicates recent infection (primary, reactivation, or reinfection). IgM positive results in combination with low IgG avidity results are considered reliable evidence for primary infection.

Primary CMV infection is treated and/or counseled differently than latent CMV infection. However, current methods to differentiate between primary and latent infection are imperfect, can yield ambiguous results for many individuals, and fail to discriminate between more and less recent primary infection, which is of high clinical utility.

In fact, routine screening for primary CMV infection during pregnancy is not recommended in the United States for several reasons. Most laboratory tests currently available to identify a first-time infection can be difficult to interpret. Current tests cannot predict if the fetus may become infected or harmed by infection. The lack of a proven treatment to prevent or treat infection of the fetus reduces the potential benefits of prenatal screening.

### BRIEF DESCRIPTION OF THE DRAWINGS

The present disclosure is illustrated by way of example and not limitation in the figures of the accompanying drawings, in which like references indicate similar elements.

FIG. 1 is a diagram illustrating an example framework to determine classifications of latent or primary infection and/or time since CMV exposure or infection, particularly for subjects classified as primary infection, including using machine learning techniques, in accordance with one or more implementations.

FIG. 2 is a diagram illustrating an example machine learning architecture that includes an optional convolutional neural network to determine classifications of latent or primary infection and/or time since CMV exposure or infection, particularly for subjects classified as primary infection, in accordance with one or more implementations.

FIG. 3A is a flow diagram illustrating a first example process to train a machine learning model for determining classifications of latent or primary infection and/or time since CMV exposure or infection, particularly for subjects classified as primary infection, in accordance with one or more implementations.

FIG. 3B is a flow diagram illustrating a first example process to determine classifications of latent or primary infection and/or time since CMV exposure or infection, particularly for subjects classified as primary infection using a trained machine learning model, in accordance with one or more implementations.

FIG. 4 illustrates a diagrammatic representation of a machine in the form of a computer system within which a set of instructions may be executed for causing the machine to perform any one or more of the methodologies discussed herein, according to an example implementation.

FIG. 5 illustrates a Uniform Manifold Approximation and Projection (UMAP) plot indicating features of individuals in which CMV is latent or primary and also indicating pregnancy status.

FIG. 6 illustrates a scatterplot of antibody features of individuals in which CMV is latent and a scatterplot indicating features of antibodies individuals in which CMV is active.

FIG. 7A illustrates a chart depicting the effects of time on the performance of an example machine learning model disclosed herein in predicting which women of subject group have a primary infection and which have a latent infection.

FIG. 7B illustrates another chart depicting the effects of time on the performance of an example machine learning model disclosed herein in predicting which women of subject group have a primary infection.

FIG. 8 illustrates a UMAP plot indicating features of individuals in which CMV is latent or primary.

FIG. 9 illustrates a chart demonstrating that IgA antibodies show elevated levels in primary infection.

FIG. 10A illustrates a chart demonstrating that latent subjects show elevated levels of complement deposition. FIG. 10B illustrates a chart demonstrating that latent subjects show phagocytosis with pentamer and gB CMV antigens.

FIG. 11 illustrates a volcano plot of antibody features and CMV antigen. Volcano plot represents the log 2 fold change (x-axis) against the −log 10 p-value calculated from the Mann Whitney test.

FIG. 12 illustrates a chart demonstrating that IgM (top panel), IgA (middle panel), and IgG (bottom panel) binding to CMV antigens tegument 1 (CG1), tegument 2 (CG2), glycoprotein B (gB), and the pentamer complex. Data are median fluorescent intensity (MFI) of Luminex measurements reported as the mean from technical replicates. Solid line indicates median. Statistics were calculated by the Mann-Whitney test. ns denotes not significant. * p<0.05, ** p<0.01, *** p<0.001, and **** p<0.0001.

FIG. 13A-13D shows information and data from exemplary modeling of primary and latent CMV infection. FIG. 13A is a confusion matrix of predicted class labels relative to ground truth. FIG. 13B depicts regression model predictions of primary or latent CMV subjects. Permuted data denotes data with mixed subject labels to examine model overfitting. Permuted data is presented as the mean for each of 100 independent permutation runs. Results are representative of one entire cross validated run that displayed average performance. FIG. 13C shows the top 10 feature importance coefficients in the model in part a. FIG. 13D shows raw data for the top 4 features from the predictive model. Statistics were calculated by the Mann-Whitney test. ns denotes not significant. * p<0.05, ** p<0.01, *** p<0.001, and **** p<0.0001.

FIG. 14 illustrates a chart depicting model validation against experimental noise.

FIG. 15A-15D shows information and data from exemplary longitudinal models.

FIG. 15A depicts regression model predictions on primary and latent longitudinal samples and plotted according to visit. FIG. 15B depicts model predictions for the primary longitudinal samples as a function of time. FIG. 15C depicts model predictions on the primary samples used for predicting days post symptom onset. Data shows the measure of the predicted label and its closeness to the true label. Days post infection (DPI). FIG. 15D depicts model longitudinal plots of Luminex binding data for top three features from the model. Black line denotes average mean over time. Statistics were calculated by the Mann-Whitney test. ns denotes not significant. * p<0.05, ** p<0.01, *** p<0.001, and **** p<0.0001.

### DETAILED DESCRIPTION

Immunological information associated with host subjects, including for example information related to anti-herpesvirus antibodies present in host subjects, can indicate latent or primary infection and/or time since CMV exposure or infection, particularly for host subjects classified as primary infection.

Information associated with a host subject that has been exposed to, infected with, and/or vaccinated against a herpesvirus, including for example information related to anti-herpesvirus antibodies present in a host subject, can indicate information about infection status (e.g., latent or primary infection and/or time since viral exposure or infection, particularly for host subjects classified as primary infection), and/or suitability for receiving a therapeutic intervention, such as antiviral therapy or a herpesvirus vaccine.

Thus, in one aspect described herein, antibody profiling data and machine learning models can be used first to categorize subjects as being in either latent or primary infection period, and then to define time since infection for subjects classified as primary infection.

Prior to the present disclosure, methods to differentiate between primary and latent infection yielded ambiguous results for many individuals and failed to discriminate between more and less recent primary infection, which is of high clinical utility. Examples disclosed herein implement one or more machine learning algorithms to generate a model that can analyze training data obtained from at least one of host subject having latent infection and/or at least one host subject having primary infection. The trained model can then be used to identify a screening subject as having latent infection or having primary infection and/or to determine time since exposure or infection. For example, systems and methods disclosed herein use machine learning algorithms to (1) determine, based on one or more immunological characteristics, whether a host has a latent herpesvirus (e.g., cytomegalovirus (CMV)) infection or a primary (e.g., cytomegalovirus (CMV)) infection; (2) determine, based on one or more immunological characteristics, time since exposure to or infection with a herpesvirus; and (3) subsequently identify and provide therapeutic intervention option(s) for such subjects. In one or more examples, the systems and methods use training data to (1) determine which characteristics result in a trained machine learning algorithm that is able to accurately distinguish between latent and primary infection and/or determine time since exposure to or infection with a herpesvirus and (2) subsequently generate such a trained machine learning algorithm. Thus, the systems and methods disclosed herein use a limited rules that are specifically designed to achieve an improved technological result of being able to distinguish between latent and primary infection and/or determine time since exposure to or infection with a herpesvirus. The structure of the limited rules reflects a specific implementation of machine learning techniques, such as convolutional neural networks, that no person in the industry would have likely utilized in the search therapeutic interventions for herpesvirus and/or other viruses.

For example, the training data can be obtained from an assay that detects immunological characteristics of subjects. In one or more illustrative examples, the assay can detect characteristics of at least one of antibodies or antigens present in a host subject having latent infection and/or a host subject having primary infection.

The training data can be analyzed using one or more machine learning techniques to determine immunological characteristics of subjects that have latent infection and immunological characteristics of subjects that have primary infection. The one or more machine learning techniques can be used to generate a trained model to classify subjects as having latent or primary infection and/or according to time since CMV exposure or infection, particularly for subjects classified as primary infection. In one or more illustrative examples, a model used to identify a subject as having latent infection or having primary infection and/or to determine time since exposure or infection can implement one or more convolutional neural networks.

By using advanced computational techniques, such as machine learning techniques, to analyze immunological data of subjects, the implementations described herein are able to identify a subject as having latent infection or having primary infection and/or to determine time since exposure or infection that are unable to be determined by conventional techniques. Additionally, the implementations described herein are directed to machine learning architectures that provide accurate results that are not achievable using other architectures. Further, by implementing machine learning architectures that are able to accurately identify a subject as having latent infection or having primary infection and/or to determine time since exposure or infection, the implementations described herein can provide healthcare practitioners with information that can be used to determine and provide treatments for such subjects to improve health outcomes.

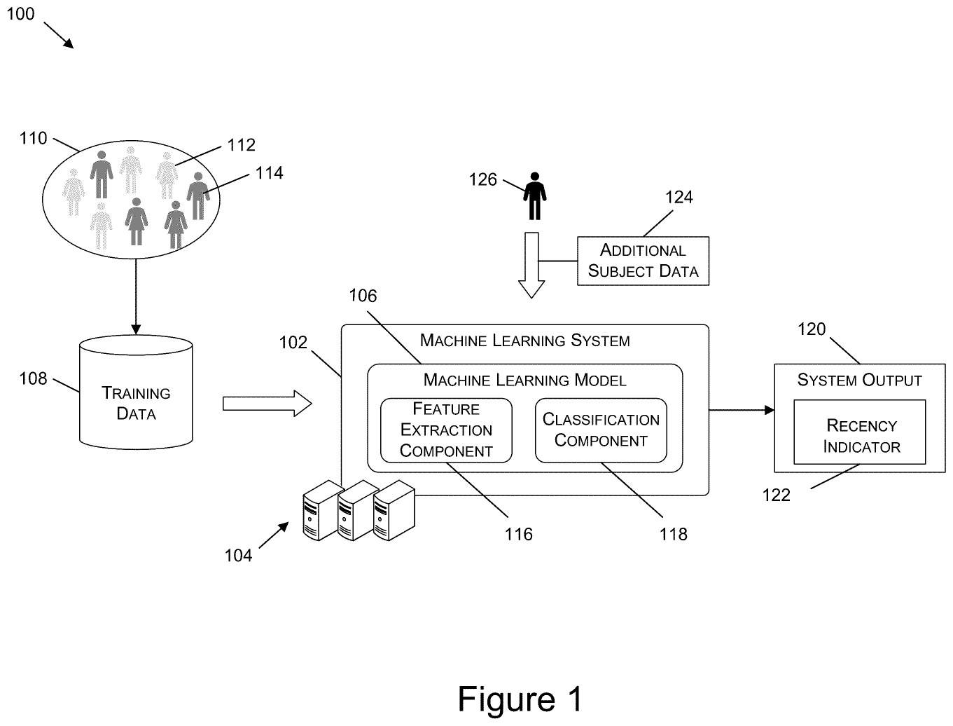

FIG. 1 is a diagram illustrating an example framework 100 to determine classifications of latent or primary infection and/or time since CMV exposure or infection, particularly for subjects classified as primary infection, using machine learning techniques, in accordance with one or more implementations. The classifications may include, for example, a latent/active classification, duration since exposure/infection classification, and/or a suitability for therapeutic intervention classification. The framework 100 can include a machine learning system 102. The machine system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In one or more implementations, at least a portion of the one or more computing devices 104 can be implemented in a distributed computing environment. For example, at least a portion of the one or more computing devices 104 can be implemented in a cloud computing architecture.

The machine learning system 102 can cause the one or more computing devices 104 to implement one or more machine learning techniques to classify subjects as latent or primary status and/or to classify subjects based on time since exposure or infection, and/or a suitability for therapeutic intervention status. The latent/active status can indicate whether a subject has a latent or a primary infection. In this regard, the classification status of a subject can be of high clinical utility as primary infection is treated/counseled differently than latent infection.

In various examples, the one or more machine learning techniques can cause the computing system 104 to at least one of learn patterns, identify features, or generate predictive information without being explicitly programmed. The machine learning system 102 can implement at least one of Logistic Regression (LR), Naive-Bayes, Random Forest (RF), neural networks (NN), matrix factorization, and Support Vector Machines (SVM) tools to determine classifications of subjects in relation to the time since exposure to or infection with a virus.

In one or more examples, the machine learning system 102 can undergo a training process that can be used to generate one or more models, such as a machine learning model 106. For example, the machine learning model 106 can be used to identify a subject as having latent infection or having primary infection and/or to determine time since exposure or infection. In various examples, the machine learning model 106 can determine a classification or category for subjects that corresponds to the status of the respective subjects as having latent or primary infection. The training data 108 can be obtained from one or more samples collected from individual subjects included in the group of subjects 110. The group of subjects 110 can include a number of humans. In one or more additional scenarios, the group of subjects 110 can include mammals different from humans. In these scenarios, the training data 108 can include genomics information, immunological data, personal information, such as age, gender, ethnic background, one or more combinations thereof, and so forth.

In one or more examples, an assay can be implemented with respect to the samples collected from the group of subjects 110 to generate the training data 108. The sample can comprise one or more bodily fluids, whole blood, platelets, serum, plasma, stool, red blood cells, white blood cells, endothelial cells, tissue derived from a subject, synovial fluid, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, fluid from nasal brushings, fluid from a pap smear, or any other bodily fluids. A bodily fluid can include saliva, blood, or serum.

At least a portion of the information obtained from the assay can be included in the training data 108. In various examples, the training data 108 can be comprised of immunological data of the group of subjects 110. The immunological data included in the training data 108 can indicate features of at least one of antigens, antibodies, or proteins involved in the immune system response that are present in the group of subjects 110. The training data 108 can also indicate the absence of at least one of antigens, antibodies, or proteins involved in the immune system response with respect to the group of subjects 110. In one or more additional examples, the training data 108 can indicate an amount of at least one of antigens, antibodies, or proteins involved in the immune system response that are present in the group of subjects 110. In one or more further examples, the training data can indicate structural changes of at least one of antigens, antibodies, or proteins involved in the immune system response that are present in the group of subjects 110. In one or more illustrative examples, the structural changes can indicate the ability of antibodies to recognize a number of viral proteins and strains and the isotypes, subclasses, and Fc receptor binding properties of antigen-specific antibodies. In one or more additional illustrative examples, the structural changes can correspond to binding of antibodies to antigens and/or other proteins involved in the immune system response to the virus that are present in the group of subjects 110. In various examples, the training data 108 can also include genomics data of the group of subjects 110. To illustrate, the training data 110 can include transcriptional profiles of the group of subjects 110.

The machine learning system 102 can implement one or more techniques to train the machine learning model 106 to accurately make predictions based on the training data 108 provided to the machine learning system 102. The group of subjects 110 can include a number of classes of subjects. For example, the group of subjects 110 can include at least a first number of subjects 112 and a second number of subjects 114. The first number of subjects 112 can be classified as having a latent infection. Additionally, the second group of subjects 114 can be classified as having a primary infection. Additionally or alternatively, the group of subjects 110 can also be sub-grouped by time since exposure to and/or infection with the virus.

During a training phase, components of the machine learning model 106 are generated based on the training data 108 to optimize the machine learning model 106 to accurately predict an output for a given input. The training data 108 can include labeled data or unlabeled data. In situations where the training data 108 includes labeled data, the machine learning system 102 can implement a supervised training process for the machine learning model 106. In scenarios where the training data 108 includes unlabeled data, the machine learning system 102 can implement an unsupervised training process for the machine learning model 106.

Additionally, in instances where the training data 108 includes a combination of labeled data and unlabeled data, the machine learning system 102 can implement a semi-supervised training process for the machine learning model 106. In one or more illustrative examples, at least a portion of the training data 108 can be labeled. For example, immunological data obtained from the first number of subjects 112 can be labeled as corresponding to latent infection and immunological data obtained from the second number of subjects 114 can be labeled as corresponding to primary infection. In these implementations, the machine learning system 102 can implement a supervised process or a semi-supervised process to train the machine learning model 106.

In various examples, during the training process of the machine learning model 106, the machine learning system 102 can optimize at least one of parameters, weights, coefficients, or other components of the machine learning model 102. In one or more illustrative examples, the machine learning system 102 can train the machine learning model 106 in order to minimize a loss function of the machine learning model 106. The loss function can be implemented to return a number representing an indicator of performance of the machine learning model 106 in mapping a validation set of the training data 108 to the correct output. In training, if the loss function value is not within a pre-determined range, based on the validation set of the training data 108, one or more techniques, such as backpropagation can be used, to modify components of the machine learning model 106, such as weights, parameters, and/or coefficients of the machine learning model 106, to increase the accuracy of the results produced by the machine learning model 106.

In one or more examples, the machine learning system 102 can implement a training process that includes a number of iterations of analyzing the training data 108 to determine components of the machine learning model 106 and validating the machine learning model 106 to determine an accuracy of classifications made by the machine learning model 106 after one or more iterations. In one or more illustrative examples, during individual iterations of the training process, the machine learning system 102 can allocate a first portion of the training data 108 to determine components of the machine learning model 106 and allocate a second portion of the training data 108 to validate the classifications generated by the machine learning model 106 using the first portion of the training data 108. The machine learning system 102 can determine a level of accuracy of the machine learning model 106 for an individual iteration of the training process based on an amount of similarity between the classifications made by the machine learning model 106 during an iteration and the classifications of the second portion of the training data 108. In scenarios where the level of accuracy corresponds to a threshold level of accuracy, the machine learning system 102 can end the training process and in situations where the level of accuracy is less than a threshold level of accuracy, the machine learning system 102 can continue the training process. In instances where the training process continues, the machine learning system 102 can modify weights, parameters, and/or other components of the machine learning model 106 in an attempt to improve the accuracy of the predictions made by the machine learning model 106 in subsequent iterations. The machine learning system 102 can produce a trained version of the machine learning model 106 after the training process is complete.

The machine learning system 102 can include a feature extraction component 118 and a classification component 118. A feature can include an individual measurable property of a phenomenon being observed. Features can be characterized in different ways. For example, a feature can be characterized numerically, graphically, or using a string of characters. In one or more examples, features can correspond to immunological characteristic of individuals. In one or more examples, during the training process, the feature extraction component 102 can analyze features of the group of subjects 110 to determine correlations of the features of the group of subjects 110 in relation to outcomes generated by the machine learning model 106.

Feature extraction is a process to reduce the amount of computing resources utilized to characterize a large set of data. When performing analysis of complex data, one of the major problems stems from the number of variables involved. Analysis with a large number of variables generally requires a large amount of memory and computational power, and it may cause a classification algorithm to overfit to training samples and generalize poorly to new samples. Feature extraction is a general term describing methods of constructing combinations of variables to get around these large data-set problems while still describing the data with sufficient accuracy for the desired purpose. In implementations described herein, the feature extraction component 116 can determine a number of immunological features that can be used to identify a subject as having latent infection or having primary infection and/or to determine time since exposure or infection.

In one or more examples, the feature extraction component 116 can analyze an initial set of the training data 108 and determine features that are informative and non-redundant with respect to classifications made by the machine learning system 102. Determining a subset of the initial features can be referred to as feature selection. The selected features are expected to contain relevant information from the input data, so that the desired outcome can be generated by the machine learning system 106 using this reduced representation instead of the complete initial data.

In various examples, the feature extraction component 116 can implement machine-learning techniques that can determine immunological features that correspond to latent vs. primary infection that conventional statistical techniques are unable to identify. In one or more illustrative examples, the feature extraction component 116 can implement a convolutional neural network that includes one or more convolutional layers to determine immunological features of the plurality of subjects 110 that are indicators for identifying a subject as having latent infection or having primary infection and/or to determining time since exposure or infection. The goal of training the one or more convolutional layers of the feature extraction component 116 is to find values of at least one of parameters, weights, or other components of the one or more convolutional layers that make them adequate for the desired task of identifying a subject as having latent infection or having primary infection and/or to determining time since exposure or infection. In one or more additional examples, one or more additional machine learning techniques can be implemented by the feature extraction component 116. To illustrate, the feature extraction component 116 can implement one or more support vector machines, one or more random forests, one or more logistic regression models, one or more feed-forward neural networks, or one or more combinations thereof.

The classification component 118 of the machine learning model 118 can obtain output from the feature extraction component 116 and determine one or more classifications based on the information obtained from the feature extraction component 116. For example, the classification component 118 can obtain values related to a set of immunological features identified by the feature extraction component 116 and determine a classification of subjects based on the values of the set of immunological features. In various examples, the classification component 118 can implement one or more fully connected layers to determine an output relating to the classification of subjects with respect to latent or primary infection and/or time since exposure to or infection with the virus.

In one or more examples, the machine learning system 102 can train and implement the machine learning model 106 to generate a system output 120. The system output 120 can include an indicator 122. The indicator 122 can correspond to identification of a subject as having latent infection or having primary infection and/or to determination of the time since exposure or infection. The indicator 122 can indicate a category of a subject as latent or primary and/or more recently exposed or infected or less recently exposed or infected. In one or more additional examples, the indicator 122 can include a numerical indicator that corresponds to the time since exposure or infection.

In still other examples, the indicator 122 can indicate two categories for subjects. To illustrate, the indicator 122 can indicate a first group of subjects having latent infection and a second group of subjects having primary infection.

In one or more illustrative examples, after training the machine learning model 106, subject data 124 of a screening subject 126 can be provided to the machine learning system 102 to determine a recency indicator 122 for the screening subject 126. The screening subject 126 is not included in the group of subjects 110 that correspond to the training data 108. The subject data 124 can include various types of information related to the screening subject 126. For example, the subject data 124 can include genomic data of the screening subject 126. The subject data 124 can also include personal information related to the screening subject 126, such as age-related data, gender-related data, health data (e.g., height, weight, blood pressure, etc.), one or more combinations thereof, and the like. Further, the subject data 124 can include immunological data related to the screening subject 126. In one or more illustrative examples, at least a portion of the subject data 124 can be obtained from one or more assays implemented with respect to one or more samples obtained from the screening subject 126. In one or more additional examples, additional information can be provided to the machine learning system 102 that corresponds to one or more potential subjects that can contract the virus from the screening subject 126.

The feature extraction component 116 can analyze the subject data 124 to determine values for the set of features identified during the training process that can be used to determine the recency indicator 122. After determining the values of the set of features included in the subject data 124, the feature extraction component 116 can perform one or more calculations with respect to the values and provide an output to the classification component 118. The classification component 118 can then perform further calculations on the output obtained from the feature extraction component 116 to generate the recency indicator 122.

In one or more examples, the features of the screening subject 126 analyzed by the machine learning model 106 can correspond to at least one of an indicator of specificity of antibodies to bind to one or more antigens or an indicator of specificity of antibodies to bind to one or more epitopes of one or more antigens. The features of the screening subject 126 analyzed by the machine learning model 106 can also indicate amounts of at least one of isotypes of antibodies or subclasses of antibodies present in the screening subject 126. Additionally, the features of the screening subject 126 analyzed by the machine learning model 106 can indicate a glycosylation profile of one or more sites of one or more antibodies present in the screening subject 126. Further, the features of the screening subject 126 analyzed by the machine learning model 106 can correspond to an indicator of affinity of antibodies to a group of fragment crystallizable (Fc) region receptor sites. In still further examples, the features of the screening subject 126 analyzed by the machine learning model 106 can correspond to an amount of activation of one or more antibody effector functions. In various examples, after analyzing immunological features of the screening subject 126 to produce a recency indicator 122 for the screening subject 126, treatment options and/or preventative measures can be identified based on the status of the screening subject 126 as having a latent infection or a primary infection and/or the time since screening subject 126 was exposed to and/or infected with the virus.

In any examples described herein, immunological features can be obtained by performing an assay with respect to a sample obtained from the subject. The immunological features obtained by performing the assay can be represented as numerical values that are subsequently fed to a trained machine learning model as a vector. In such examples, the immunological features or other information represented as numerical values to facilitate feeding of such information to the trained machine learning model as part of an input vector.

In one or more examples, the immunological features can include information related to at least one of one or more antibodies, one or more antigens, one or more additional proteins, or one or more combinations thereof. For example, the immunological features can include measures of epitope specificity with respect to one or more antibodies (e.g., anti-herpesvirus or anti-CMV antibodies) present in the subject. To illustrate, the immunological features can include measures of antibody recognition of one or more antigens. In one or more additional examples, the immunological features can include measures of at least one of folding or unfolding with respect to one or more antigens present in the subject.

The immunological features can also indicate at least one of isotypes or subclasses of antibodies present in the subject. In various examples, the immunological features can indicate a measure of at least one of IgA antibodies present in the subject, IgA1 antibodies present in the subject, or IgA2 antibodies present in the subject. Additionally, the immunological features can indicate a measure of at least one of IgG antibodies present in the subject, IgG1 antibodies present in the subject, IgG2 antibodies present in the subject, IgG3 antibodies present in the subject, or IgG4 antibodies present in the subject. Further, the immunological features can indicate a measure of IgM antibodies present in the subject.

In addition, the immunological features can indicate a measure of binding of antibody Fc regions to antigens present in the subject. For example, the immunological features can indicate a measure of binding of Fc receptor sites, such as FcγR receptor sites. In one or more implementations, the immunological feature can indicate a measure of the characteristics of antibody Fc regions belonging to antigen-specific antibodies. In one or more examples, the immunological features can indicate a measure of binding of fragments of antibody variable regions (Fv) of at least one of heavy chains or light chains of antibodies present in the subject. To illustrate, the immunological features can indicate an amount of binding of Fv binding to cytomegalovirus glycoprotein B. The amount of binding of antibodies of the subject to cytomegalovirus glycoprotein B can correspond to an amount of folding or unfolding of cytomegalovirus glycoprotein B molecules present in the subject. In one or more illustrative examples, the immunological features subject can also indicate a measure of IgM antibody in the pentamer state binding antigens present in the subject. Further, the immunological features subject can indicate a measure of binding to at least one of tegument 1 or tegument 2 of the virus 306 by Fv regions of antibodies present in the subject. In various examples, the amount of binding of Fv regions of antibodies of the subject to tegument 1 or tegument 2 of the virus 306 can correspond to an amount of folding or unfolding with respect to tegument 1 or tegument 2 of the virus present in the subject.

The immunological features can also indicate glycosylation profile of antibodies present in the subject. The glycosylation profile of an antibody can indicate identity and prevalence of glycans incorporated at N-glycosylation sites of the antibody. In one or more examples, the immunological features can indicate activity of functions related to antibodies present in the subject. The functions indicated by the immunological features can include at least one of neutralization, phagocytosis by monocytes, phagocytosis by neutrophils, complement deposition, or natural killer (NK) cell activation.

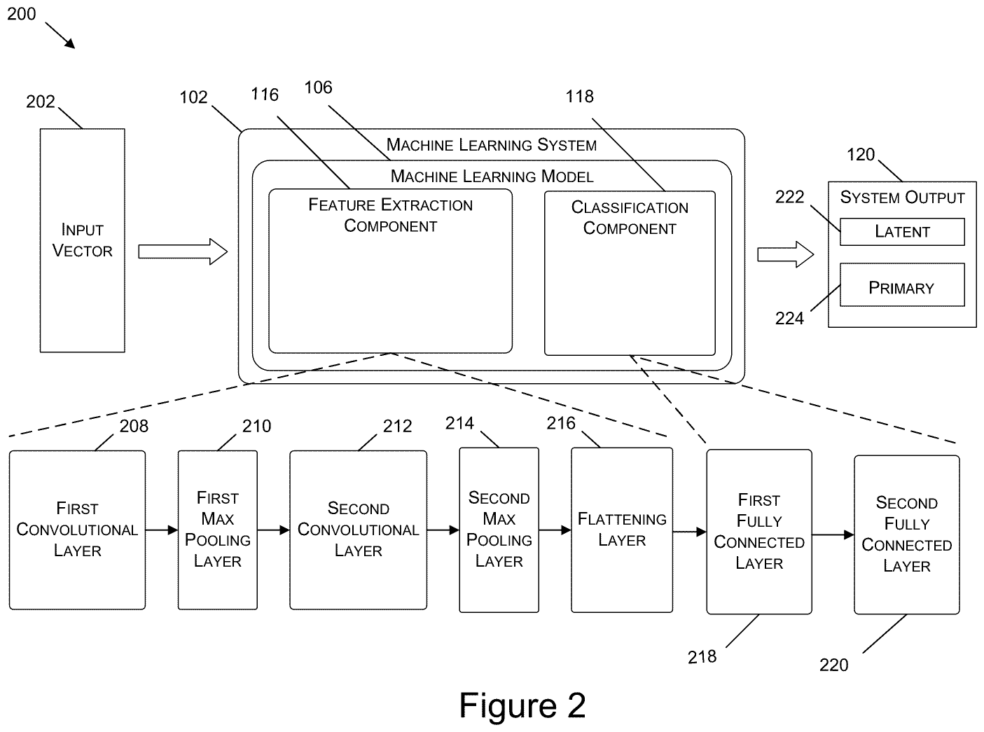

FIG. 2 is a diagram illustrating an example machine learning architecture 200 that includes a convolutional neural network to determine classifications of latent or primary infection and/or time since CMV exposure or infection, particularly for subjects classified as primary infection, in accordance with one or more implementations. The machine learning architecture 200 can include the machine learning system 102. In the illustrated example, the machine learning model 106 of the machine learning system 102 is a convolutional neural network; however, other machine learning algorithms may be employed. The machine learning system 102 can obtain an input vector 202 that includes data that is processed by the feature extraction component 116 and the classification component 118 of the machine learning model 106 to generate a system output 120 that indicates latent or primary infection and/or time since CMV exposure or infection.

The input vector 202 can include numerical values representative of immunological features corresponding to a subject that are provided to the machine learning system 102. In various examples, the input vector 202 can include values of thousands of immunological features. For example, in situations where the input vector 202 includes transcriptomic data, the input vector 202 can include up to tens of thousands of values. In one or more additional examples, the input vector 202 can include values of at least 5 immunological features, at least 10 immunological features, at least 20 immunological features, at least 32 immunological features, values of at least 64 immunological features, values of at least 96 immunological features, values of at least 128 immunological features, values of at least 164 immunological features, values of at least 192 immunological features, or values of at least 224 immunological features. In one or more illustrative examples, the input vector 202 can include values from 140 to 180 immunological feature, values from 150 to 200 immunological features, or values from 100 to 150 immunological features.

The feature extraction component 116 of a convolutional neural network can include a number of convolutional layers. Convolutional layers of the convolutional neural network can include differing numbers of filters in at least some implementations. In one or more additional examples, at least two of the convolutional layers can have a same number of filters. In one or more examples, individual filters of the convolutional layers of the convolutional neural network can include a matrix of numerical values that are applied to the values of the input vector 202. The numerical values of the filters of the convolutional neural network can be determined during a training process of the machine learning model 106. The convolutional layers of the convolutional neural network can generate respective feature maps. In various examples, the feature extraction component 116 of the convolutional neural network can include a number of pooling layers that reduce the size of the feature maps produced by the convolutional layers. In one or more illustrative examples, the pooling layers of the convolutional neural network can include max pooling layers. In one or more additional illustrative examples, the pooling layers of the convolutional neural network can include average pooling layers.

Additionally, the feature extraction component 116 of the convolutional neural network can implement one or more normalization techniques, one or more activation techniques, or both one or more normalization techniques and one or more activation techniques. In one or more additional illustrative examples, the one or more normalization techniques can include batch normalization or local normalization. In one or more further illustrative examples, the one or more activation techniques can implement one or more rectifier linear unit (ReLU) functions. The feature extraction component 116 of the convolutional neural network can also include one or more flattening layers to generate an output that can be provided to the classification component 118. In various examples, the one or more flattening layers can generate a one-dimensional vector that is provided to the classification component 118.

The classification component 118 of a convolutional neural network can include a number of fully connected layers. The number of fully connected layers can include a feed forward neural network. Individual fully connected layers can include a number of neurons with each neuron of one fully connected layer connected to each neuron of an additional fully connected layer. The classification component 118 can implement one or more activation functions to determine a classification that is included in the system output 120.

In the illustrative example of FIG. 2, the feature extraction component 116 of the convolutional neural network can include a first convolutional layer 208 followed by a first max pooling layer 210. In one or more illustrative examples, the first convolutional layer 208 can include 32 filters. The first max pooling layer 210 can be followed by a second convolutional layer 212 that is, in turn, followed by a second max pooling layer 214. In one or more additional illustrative examples, the second convolutional layer 212 can include a greater number of filters than the first convolutional layer 208. To illustrate, the second convolutional layer 212 can include 64 filters. The second max pooling layer 214 can be followed by a flattening layer 216. In various examples, a ReLU function can be applied to the feature maps generated by the first convolutional layer 208 and the second convolutional layer 212.

The output of the flattening layer 216 can be provided to a first fully connected layer 218 that is coupled to a second fully connected layer 220. The classification component 118 can implement a SoftMax function with respect to the fully connected layers 218, 220 to determine a probability for the input vector 202 to correspond to a plurality of classifications. For example, the classification component 118 can implement a SoftMax function with respect to the fully connected layers 218, 220 to determine a probability of the input vector 202 corresponding to a first classification 222 indicating that a subject has a latent infection and a probability of the input vector 202 corresponding to the second classification 224 indicating that the subject has a primary infection. [0066].

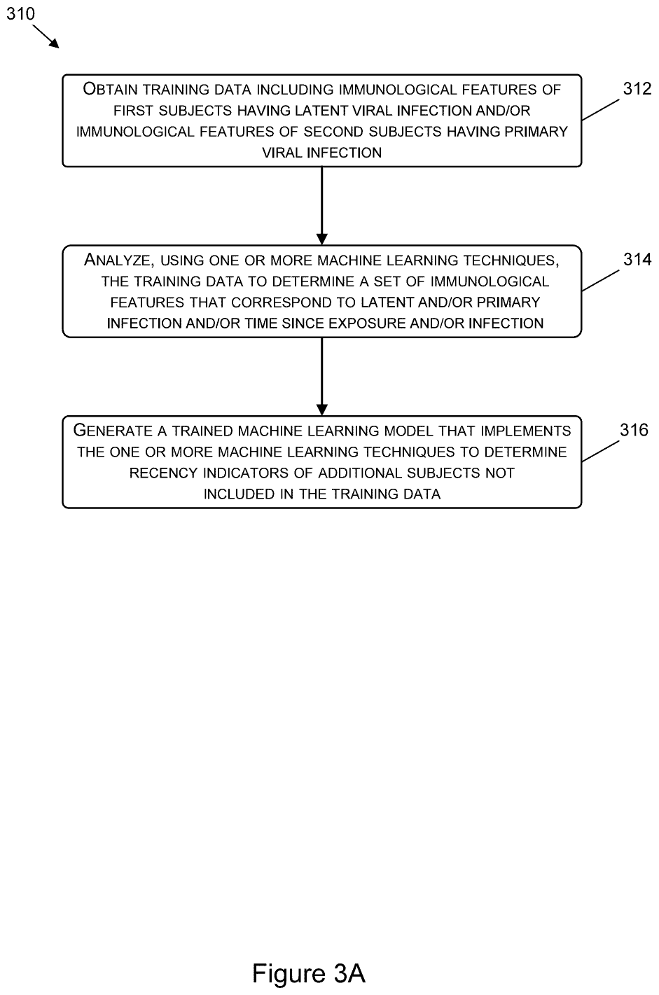

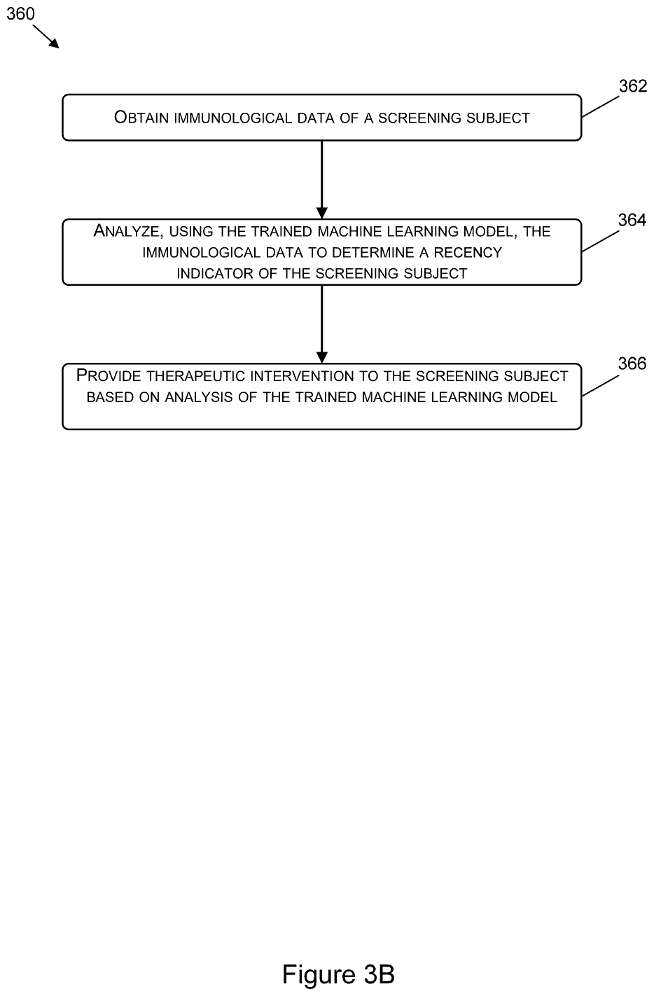

FIGS. 3A and 3B illustrate example processes related to the classification of latent or primary infection and/or time since CMV exposure or infection using machine learning techniques. The example processes are illustrated as a collection of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the processes.

FIG. 3A is a flow diagram illustrating a first example process 310 to train a machine learning model for determining classifications of latent or primary infection and/or time since CMV exposure or infection, in accordance with one or more implementations. At 312, the process 310 includes obtaining training data including immunological features of first subjects having latent viral infection and/or immunological features of second subjects having primary viral infection.

The process 310 also includes, at 314, analyzing, using one or more machine learning techniques, the training data to determine a set of immunological features that correspond to latent and/or primary infection and/or time since exposure and/or infection. In addition, at 316, the process 310 includes generating a trained machine learning model that implements the one or more machine learning techniques to determine recency indicators of screening subjects not included in the training data. In one or more examples, the training process can be performed to minimize a loss function of the trained machine learning model.

FIG. 3B is a flow diagram illustrating a first example process 360 to determine determining classifications of latent or primary infection and/or time since CMV exposure or infection using a trained machine learning model, such as the machine learning model generated at block 316, in accordance with one or more implementations. At 362, the process 360 includes obtaining immunological data (e.g., additional or third immunological data) of an screening subject. The immunological data can indicate values of the set of immunological features for the screening individual.

At 364, the process 360 includes analyzing, using the trained machine learning model, the immunological data of the screening individual to determine a recency indicator of the additional subject. In one or more examples, the trained machine learning model used at 364 was generated at 316 of the process 310. In one or more other examples, the trained machine learning model used at 364 was generated via other process(es). Further, in one or more examples, the values of the set of immunological features of the immunological data of the screening individual can be included in an input vector that is provided to the trained machine learning model. The recency indicator can correspond to latent or primary infection and/or time since CMV exposure or infection.

At 366, the process 360 can include providing therapeutic intervention(s) to the screening subject in response to the system output of the trained machine learning model indicating that the additional subject has a latent or primary infection and/or based on the time since CMV exposure or infection. Therapeutic intervention option(s), such as antiviral therapy and/or administration of a vaccine, are provided to the screening subject.

In various examples, the trained machine learning model can include a feature extraction component and a classification component. In one or more examples, the feature extraction component can include a convolutional neural network having one or more convolutional layers and one or more max pooling layers. In at least some examples, the second convolutional layer can have a greater number of filters than the first convolutional layer. In one or more illustrative examples, the convolutional neural network can include a first convolutional layer having from 24 filters to 48 filters and a second convolutional layer having from 48 filters to 96 filters. The feature extraction component can implement a rectified linear unit (ReLU) activation function. The feature extraction component can also include a flattening layer that provides output of the features extraction component as input to the classification component. The classification component can include a number of fully connected layers. Further, the extraction component can implement a SoftMax function. In one or more illustrative examples, the machine learning model can include a first convolutional layer coupled to a first max pooling layer and a second convolutional layer coupled to the first max pooling layer and coupled to a second max pooling layer. The machine learning model can also include a flattening layer coupled to the second max pooling layer. Additionally, the machine learning model can include a first fully connected layer coupled to the flattening layer and a second fully connected layer coupled to the first fully connected layer. Further, the machine learning model can include a SoftMax function that generates the viral transmission indicator based on output from the second fully connected layer.

In one or more examples, an assay can be performed to obtain the first immunological data, the second immunological data, and the third immunological data. In one or more examples, assays are performed according to the techniques described in Brown, Eric P., et al. “Optimization and qualification of an Fc Array assay for assessments of antibodies against HIV-1/SIV.” Journal of immunological methods 455 (2018): 24-33; Brown, Eric P., et al. “Multiplexed Fe array for evaluation of antigen-specific antibody effector profiles.” Journal of immunological methods 443 (2017): 33-44; Ackerman, Margaret E., et al. “A robust, high-throughput assay to determine the phagocytic activity of clinical antibody samples.” Journal of immunological methods 366.1-2 (2011): 8-19; Karsten, Christina B., et al. “A versatile high-throughput assay to characterize antibody-mediated neutrophil phagocytosis.” Journal of immunological methods 471 (2019): 46-56; Butler, Savannah E., et al. “Distinct features and functions of systemic and mucosal humoral immunity among SARS-CoV-2 convalescent individuals.” Frontiers in immunology 11 (2021): 618685; and Goldberg, Benjamin S., et al. “Revisiting an IgG Fc Loss-of-Function Experiment: the Role of Complement in HIV Broadly Neutralizing Antibody b12 Activity.” Mbio 1.5 (2021): e01743-21; all of which are incorporated by reference herein in their entirety.

In various examples, the first immunological data, the second immunological data, and the third immunological data can indicate a presence or an absence of a set of antibodies that are produced by subjects in response to the virus. Additionally, the set of immunological features can correspond to at least one of isotypes or subclasses of antibodies present in subjects in which the virus is present. Further, the set of immunological features can correspond to a glycosylation profile of antibodies present in subjects in which the virus is present. In one or more illustrative examples, the set of immunological features corresponds to a level of effector functions present in subjects in which the virus is present. In one or more additional illustrative examples, the set of immunological features can correspond to at least one of a measure of folding or a measure of unfolding of antigens present in subjects in which the virus is present. In one or more further illustrative examples, the set of immunological features indicates a specificity of antibodies present in subject in which the virus is present with respect to at least one of one or more antigens or one or more epitopes of antigens present in subjects in which the virus is present.

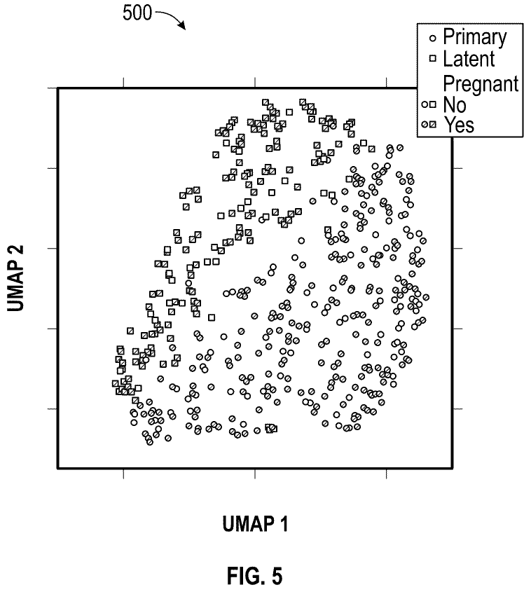

FIG. 5 illustrates a Uniform Manifold Approximation and Projection (UMAP) plot 500 indicating features of individuals in which CMV is latent or primary and also indicating pregnancy status. There was a clear distinction according to infection status.

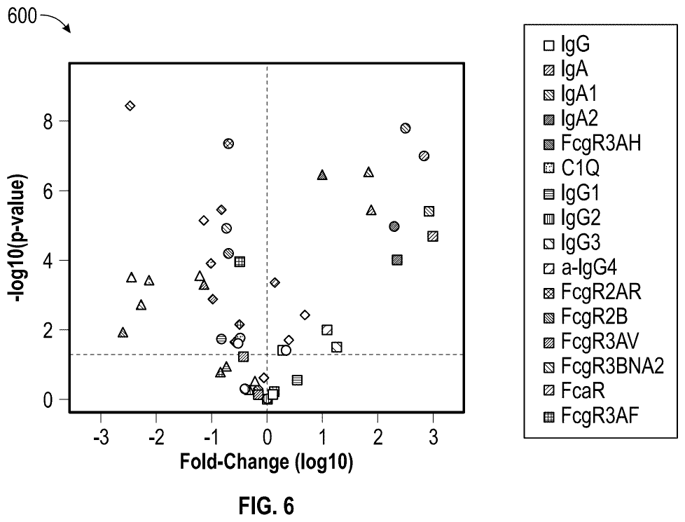

FIG. 6 illustrates a scatterplot 600 of antibody features of individuals in which CMV is latent and a scatterplot indicating features of antibodies individuals in which CMV is active.

Supervised, classical machine learning modeling was applied to latent vs. primary classification. Classification accuracy was determined using a 5-fold cross validation framework, repeated 100 times. Permuted data had scrambled labels and was then used for training/testing as an analytical negative control. Primary/non-primary status models were accurate, confident, and robust. Classical machine learning models can distinguish primary from latent CMV infection.

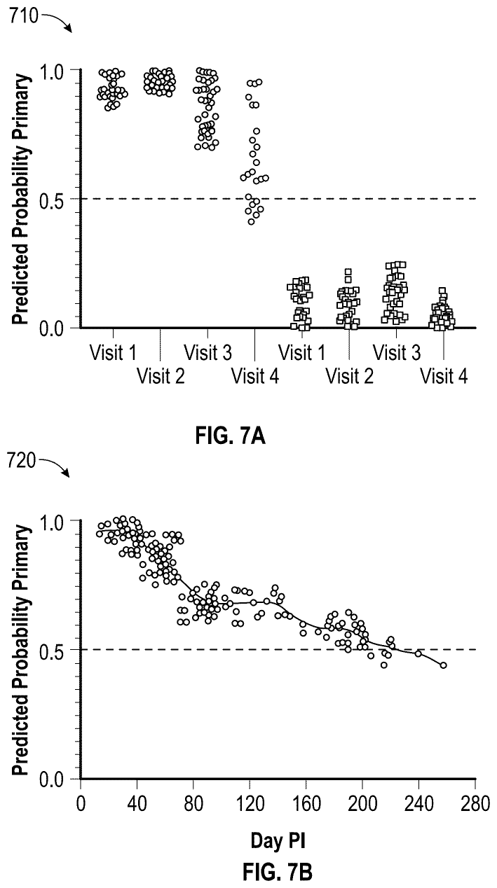

FIG. 7A illustrates a chart 710 depicting the effects of time on the performance of an example machine learning model in predicting which women of subject group of longitudinal samples have a primary infection and which have a latent infection. The model was trained on cross-sectional samples and accurately predicted the infection status of the longitudinal samples. The subject group includes 40 women with a primary infection and 37 women with a latent infection for visit 1, 40 women with a primary infection and 37 women with a latent infection for visit 2, 40 women with a primary infection and 37 women with a latent infection for visit 3, and 24 women with a primary infection and 37 women with a latent infection for visit 4.

FIG. 7B illustrates another chart 720 depicting the effects of time on the performance of an example machine learning model in predicting which women of subject group have a primary infection. The chart 720 may enable a time of infection to be calculated for a woman.

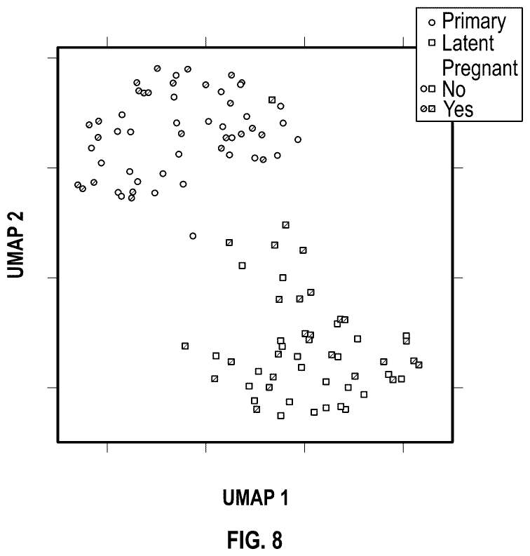

As shown in FIG. 8, antibody profiles are highly distinct between primary and latent infection. A multivariate unsupervised approach took into account multiplex antibody profiles (excluding IgM). UMAP analysis across all features clusters subjects into primary and latent infection.

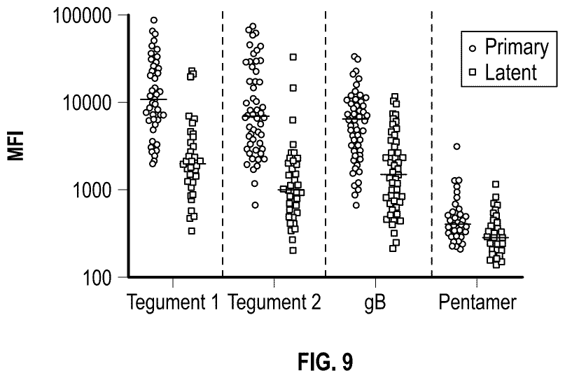

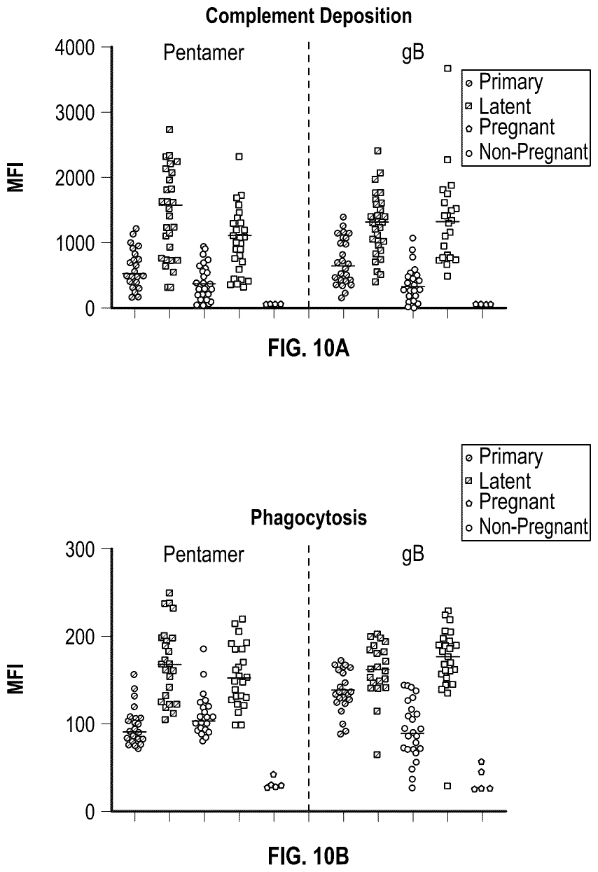

Determination of CMV infection status with novel antibody features was possible. See FIGS. 6, 9, 10A, and 10B. IgA antibodies showed elevated levels in primary infection. Fcγ receptors display elevated levels of binding to CMV antigens in latent subjects. Latent subjects show elevated levels of complement deposition and phagocytosis with pentamer and gB CMV antigens.

Distinctions between primary and latent infection are conserved between pregnant and non-pregnant women. Distinctions between pregnant and non-pregnant women are more rare/minor. There are many aspects of the antibody response that differ between primary and latent infection beyond IgM and IgG avidity. Systematic antibody profiling as described herein provides a molecular clock of time since infection that improves upon IgM/IgG avidity.

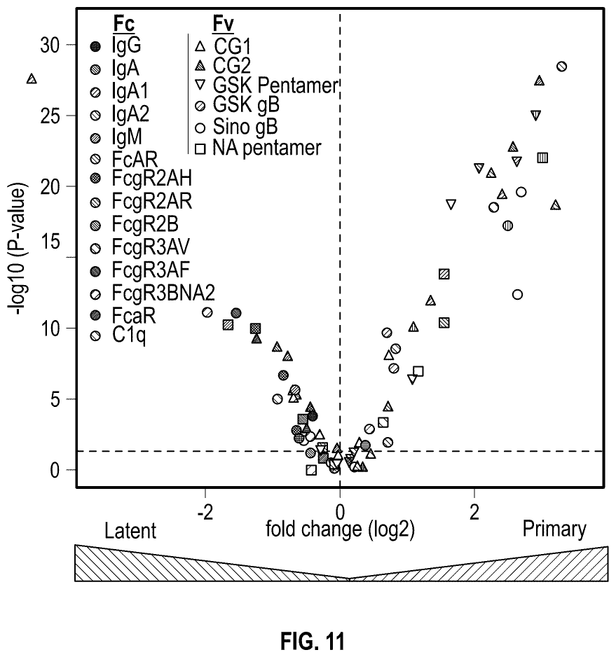

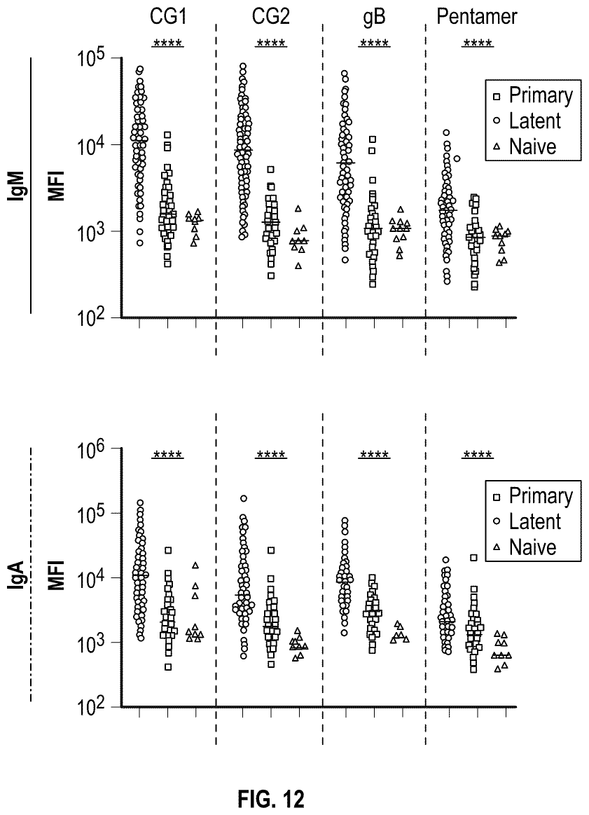

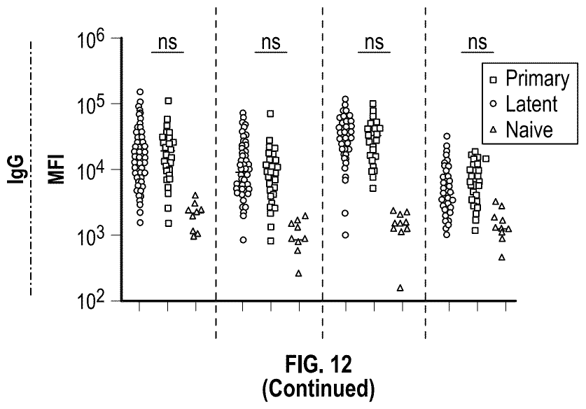

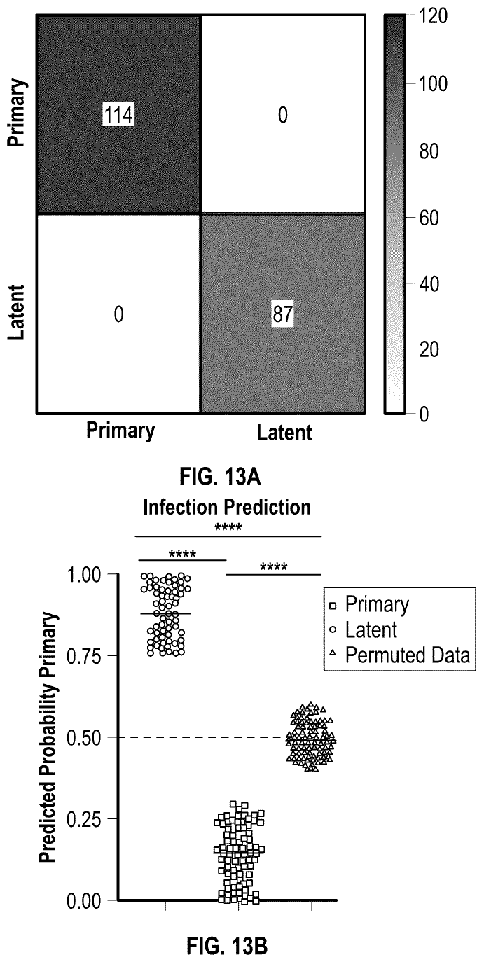

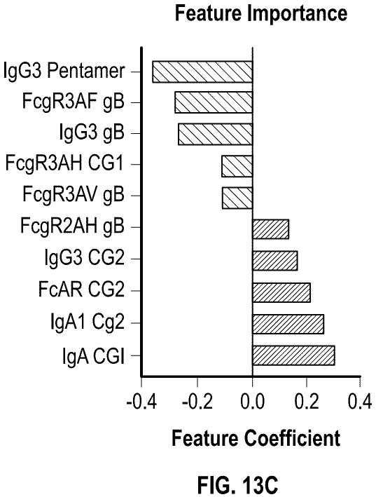

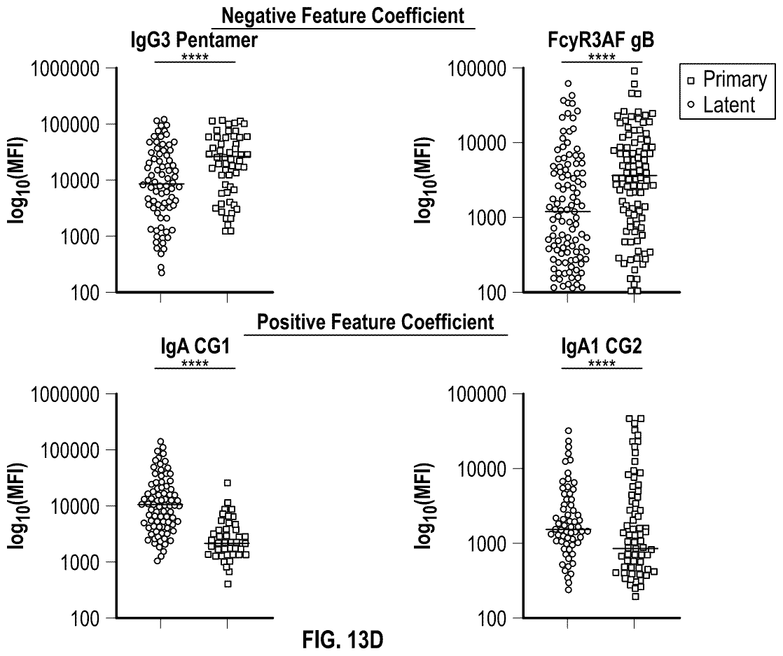

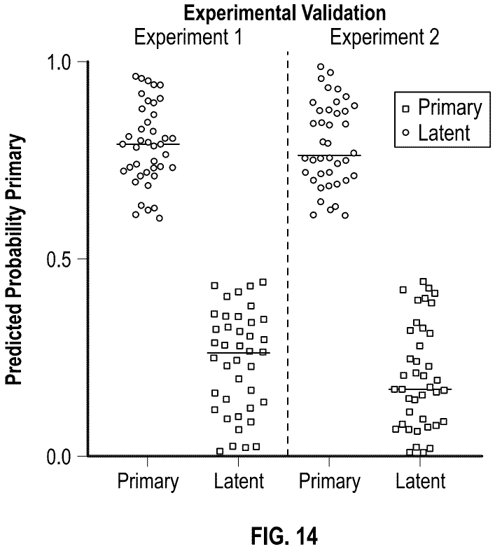

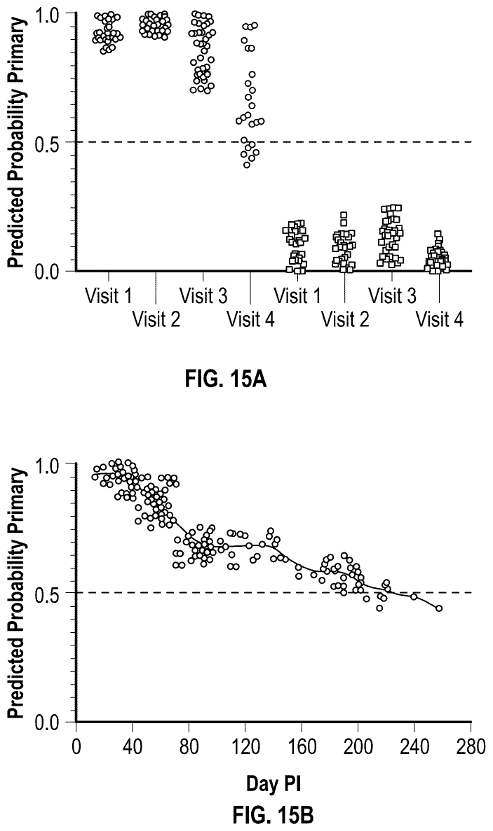

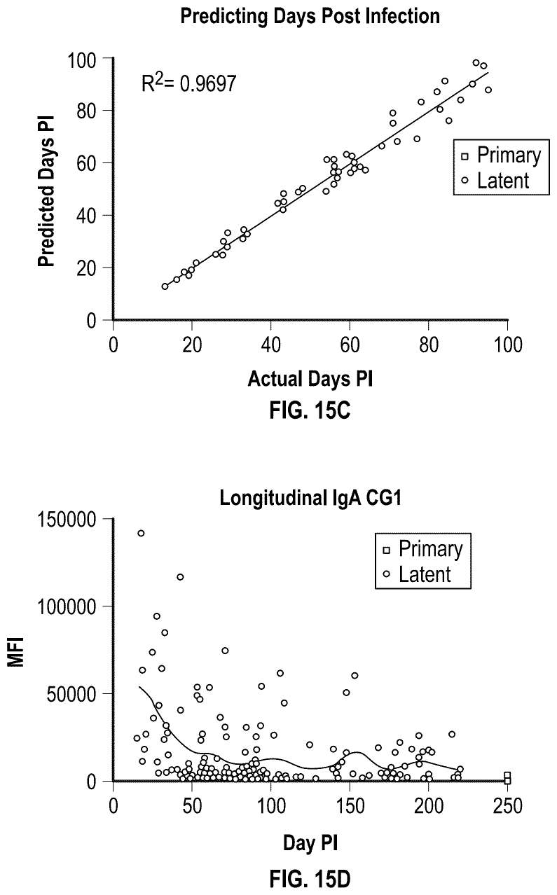

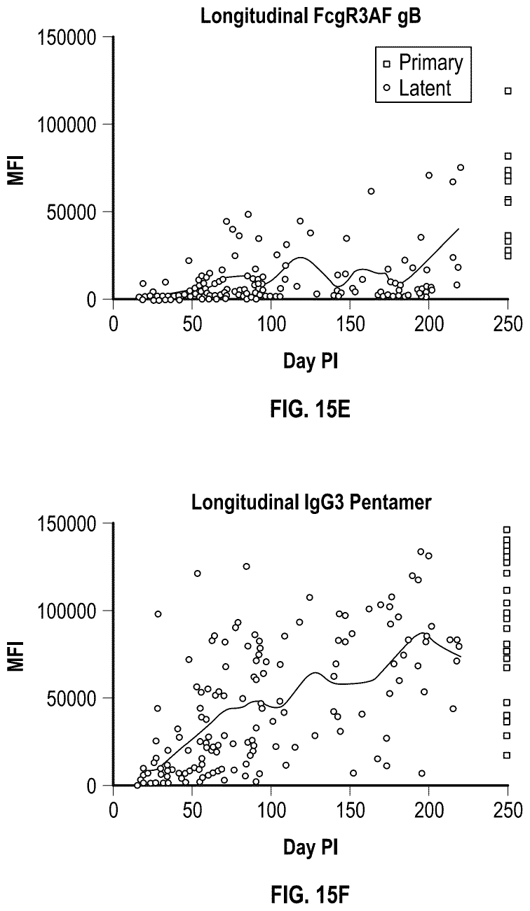

As one example, a predictive model for distinguishing CMV infection status was established. A regularized regression model was trained on 80% of data and tested on 20%. A 10-fold cross validation was performed 100 times for accuracy determination. Permuted data was created by mixing the class labels and tested in the model to provide additional information on robustness. Data was additionally used in the test set that is the same subjects from two distinct experiments to measure the model's ability to deal with experimental noise. Top model features were further explored to gain insight into the biological implications of CMV predictions. See FIGS. 11, 12, 13A-13D, 14, 15. FIG. 14 illustrates a chart depicting model validation against experimental noise. The same subjects were run in experiments over 1 year apart using different sets of reagents and run by different scientists.

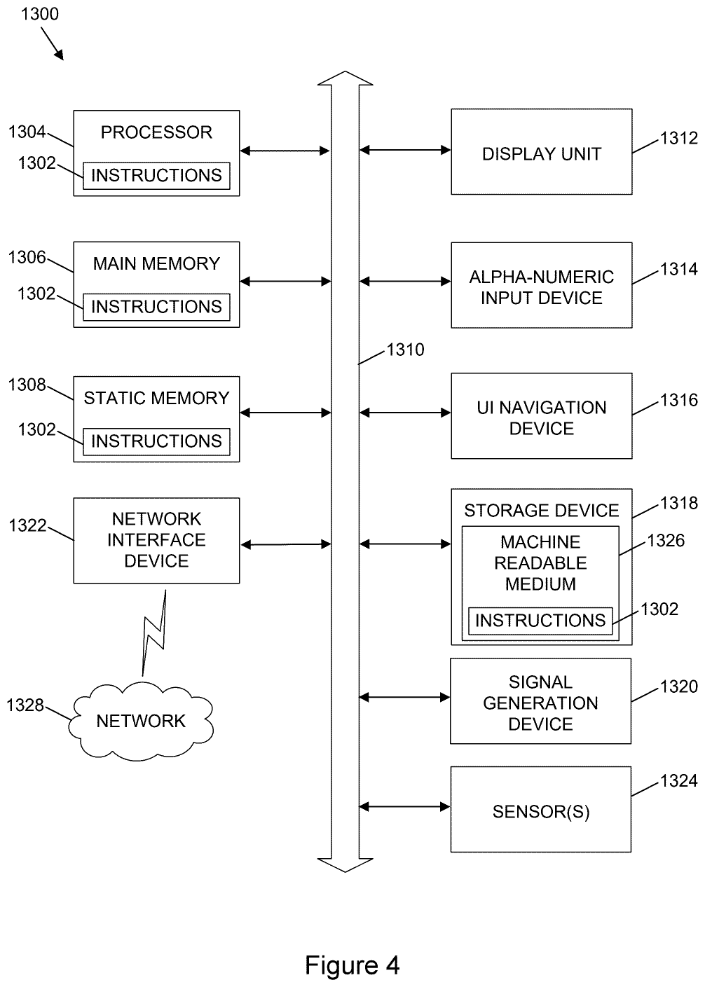

FIG. 4 illustrates a diagrammatic representation of a computing device 1300 in the form of a computer system within which a set of instructions may be executed for causing the computing device 1300 to perform any one or more of the methodologies discussed herein, according to an example, according to an example implementation. Specifically, FIG. 4 shows a diagrammatic representation of the computing device 1300 in the example form of a computer system, within which instructions 1302 (e.g., software, a program, an application, an applet, an app, or other executable code) for causing the computing device 1300 to perform any one or more of the methodologies discussed herein may be executed. For example, the instructions 1302 may cause the computing device 1300 to implement the frameworks 100, 200, described with respect to FIGS. 1 and 2, respectively, and to execute the methods 310, 360 described with respect to FIGS. 3A and 3B, respectively.

The instructions 1302 transform the computing device 1300 into a particular device programmed to carry out the described and illustrated functions in the manner described. In alternative implementations, the computing device 1300 operates as a standalone device or may be coupled (e.g., networked) to other machines. In a networked deployment, the computing device 1300 may operate in the capacity of a server machine or a client machine in a server-client network environment, or as a peer machine in a peer-to-peer (or distributed) network environment. The computing device 1300 may comprise, but not be limited to, a server computer, a client computer, a personal computer (PC), a tablet computer, a laptop computer, a netbook, a smart phone, a mobile device, other smart devices, a web appliance, a network router, a network switch, a network bridge, or any machine capable of executing the instructions 1302, sequentially or otherwise, that specify actions to be taken by the computing device 1300. Further, while only a single computing device 1300 is illustrated, the term “machine” shall also be taken to include a collection of computing devices 1300 that individually or jointly execute the instructions 1302 to perform any one or more of the methodologies discussed herein.

Examples of computing device 1300 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a processor configured via software, the processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In implementations in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example implementations (e.g., apparatus, systems, or methods) can be implemented in digital electronic circuitry, in computer hardware, in firmware, in software, or in any combination thereof. Example implementations can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a standalone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., computing device 800) and software architectures that can be deployed in example implementations.

In an example, the computing device 1300 can operate as a standalone device or the computing device 1300 can be connected (e.g., networked) to other machines.

In a networked deployment, the computing device 1300 can operate in the capacity of either a server or a client machine in server-client network environments. In an example, computing device 1300 can act as a peer machine in peer-to-peer (or other distributed) network environments. The computing device 1300 can be a personal computer (PC), a tablet PC, a network router, switch or bridge, or any machine capable of executing instructions (sequential or otherwise) specifying actions to be taken (e.g., performed) by the computing device 1300. Further, while only a single computing device 1300 is illustrated, the term “computing device” shall also be taken to include any collection of machines that individually or jointly execute a set (or multiple sets) of instructions to perform any one or more of the methodologies discussed herein.

Example computing device 1300 can include a processor 1304 (e.g., a central processing unit CPU), a graphics processing unit (GPU) or both), a main memory 1306 and a static memory 1308, some or all of which can communicate with each other via a bus 1310. The computing device 1300 can further include a display unit 1312, an alphanumeric input device 1314 (e.g., a keyboard), and a user interface (UI) navigation device 1316 (e.g., a mouse). In an example, the display unit 1312, input device 1314 and UI navigation device 1316 can be a touch screen display. The computing device 1300 can additionally include a storage device (e.g., drive unit) 1318, a signal generation device 1320 (e.g., a speaker), a network interface device 1322, and one or more sensors 1324, such as a global positioning system (GPS) sensor, compass, accelerometer, or another sensor.

The storage device 1318 can include a machine readable medium 1326 on which is stored one or more sets of data structures or instructions 1302 (e.g., software) embodying or utilized by any one or more of the methodologies or functions described herein. The instructions 1302 can also reside, completely or at least partially, within the main memory 1306, within static memory 1308, or within the processor 1304 during execution thereof by the computing device 1300. In an example, one or any combination of the processor 1304, the main memory 1306, the static memory 1308, or the storage device 1318 can constitute machine readable media.

While the machine readable medium 1326 is illustrated as a single medium, the term “machine readable medium” can include a single medium or multiple media (e.g., a centralized or distributed database, and/or associated caches and servers) that configured to store the one or more instructions 1302. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1302 can further be transmitted or received over a communications network 1328 using a transmission medium via the network interface device 1322 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As used herein, a component, such as the feature extraction component 116 and the classification component 118 can refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

In certain embodiments, any method disclosed herein may comprise the step of obtaining immunological data representative of one or more immunological features of a subject. In some such embodiments, the immunological features comprise features of antibodies present in a biological sample obtained from a subject. For example, antibody features may include the isotype and/or subclass of antibodies present in the biological sample. Additionally, antibody features may include glycosylation profile of antibodies present in the biological sample. As a further example, antibody features may include functional properties of antibodies present in the biological sample including, but not limited to, their Fc receptor binding capacity, their viral neutralization capabilities, and their ability to mediate effector functions.

In certain embodiments, any method disclosed herein may comprise the step of providing a bodily fluid sample from a subject, preferably a human subject. In some such embodiments, the subject is a pregnant subject. The bodily fluid sample may be, for example, a blood sample, such as a plasma or serum sample.

In certain embodiments, any method disclosed herein may comprise the step of detecting in a bodily fluid sample obtained from a subject a set of immunological features. In some such embodiments, the set of immunological features comprises a set of antibody features. Exemplary virus-specific antibody features include the antigen-binding specificity of antibodies present in the biological sample, the isotype and/or subclass of antibodies present in the biological sample, and the functional properties of antibodies present in the biological sample including, but not limited to, their Fc receptor binding capacity, their viral neutralization capabilities, and their ability to mediate effector functions. In some such embodiments, the set of antibody features comprises at least one of the following: antigen-binding specificity, isotype, subclass, Fc receptor binding capacity, viral neutralization, or effector function. In some such embodiments, the set of antibody features is derived from virus-specific antibodies in the bodily fluid sample. For example, an exemplary method comprises detecting in a bodily fluid sample obtained from a subject a set of anti-herpesvirus antibody features. An exemplary herpesvirus is CMV. As such, the set of antibody features may be derived from CMV-specific antibodies in the bodily fluid sample. Exemplary CMV antigens include glycoprotein B (gB), CMV pentamer complex (which is composed of glycoprotein H (gH), glycoprotein H (gL), glycoprotein UL128, glycoprotein UL130, and glycoprotein UL131A), and CMV tegument proteins such as phosphoprotein 65 (pp65). Thus, the set of antibody features may be derived from anti-gB antibodies in the bodily fluid sample and/or anti-pentamer antibodies in the bodily fluid sample. In some such embodiments, the set of anti-herpesvirus antibody features comprises at least one of the following: isotype, subclass, Fc receptor binding capacity, viral neutralization, or effector function.

In certain embodiments, any method disclosed herein may comprise the step of detecting a set of anti-herpesvirus antibody features in a bodily fluid sample from a subject, preferably a human subject. In some such embodiments, the subject is a pregnant subject. The bodily fluid sample may be, for example, a blood sample, such as a plasma or serum sample.

In some such embodiments, the anti-herpesvirus antibody feature is derived from anti-CMV antibodies in the bodily fluid sample. For example, such anti-CMV antibodies may specifically recognize a CMV protein, such as a CMV surface protein or a CMV structural protein. In some such embodiments, the anti-CMV antibodies specifically recognize CMV glycoprotein B (gB), CMV pentamer complex (which is composed of glycoprotein H (gH), glycoprotein H (gL), glycoprotein UL128, glycoprotein UL130, and glycoprotein UL131A), or a CMV tegument protein (e.g., phosphoprotein 65 (pp65)). Such anti-CMV antibodies can be identified by contacting the bodily fluid sample from the subject with a CMV protein or fragment thereof to form an antibody-protein complex between the anti-CMV antibodies present in the bodily fluid sample and the CMV protein or fragment thereof.

In some such embodiments, the set of anti-herpesvirus antibody features comprises at least one of the following: isotype, subclass, Fc receptor binding capacity, viral neutralization, or effector function.

In some such embodiments, the isotype feature represents the presence and/or amount of at least one immunoglobulin (Ig) isotype and/or subclass. Exemplary Ig isotypes include IgA, IgG, and IgM as well as IgD and IgE. The isotype feature can be determined using an antibody isotype assay. Typically, an antibody isotype assay comprises the use of specific anti-Ig antibodies capable of detecting different isotypes (and, optionally, subclasses) of antibodies present in the sample. An exemplary antibody isotype assay is an immunoassay such as an enzyme-linked immunosorbent assay (ELISA).

In some such embodiments, the isotype feature comprises presence and/or amount of IgA, IgG, and IgM. In some such embodiments, the isotype feature includes presence and/or amount of at least one of IgA, IgG, and IgM. In some such embodiments, the isotype feature includes presence and/or amount of at least two of IgA, IgG, and IgM. In some such embodiments, the isotype feature does not comprise IgA presence and/or amount. In some such embodiments, the isotype feature does not comprise IgG presence and/or amount. In some such embodiments, the isotype feature does not comprise IgM presence and/or amount.

There are four subclasses of IgG in humans: IgG1, IgG2, IgG3, and IgG4; and two subclasses of IgA in humans: IgA1 and IgA2.

In some such embodiments, the subclass feature comprises presence and/or amount of IgG1, IgG2, IgG3, and IgG4. In some such embodiments, the subclass feature includes presence and/or amount of at least one of IgG1, IgG2, IgG3, and IgG4. In some such embodiments, the subclass feature includes presence and/or amount of at least two of IgG1, IgG2, IgG3, and IgG4. In some such embodiments, the subclass feature includes presence and/or amount of IgG3 and IgG4.

In some such embodiments, the subclass feature comprises presence and/or amount of IgA1 and IgA2. In some such embodiments, the subclass feature includes presence and/or amount of at least one of IgA1 and IgA2. In some such embodiments, the subclass feature does not comprise IgA1 presence and/or amount. In some such embodiments, the subclass feature does not comprise IgA2 presence and/or amount.

In some such embodiments, the Fc receptor binding capacity feature represents the affinity of the anti-herpesvirus antibodies in the bodily fluid sample for specific Fc receptors (e.g., FcγR), such as FcγR1, FcγR2, and FcγR3. The Fc receptor binding capacity feature can be determined using, for example, an immunoassay (e.g., ELISA), flow cytometry, surface plasmon resonance (SPR), or biolayer interferometry (BLI). In some such embodiments, the Fc receptor binding capacity feature comprises affinity of the anti-herpesvirus antibodies in the bodily fluid sample for FcγR. Fc receptor binding capacity can be assessed for FcγR1 (CD64), FcγR2 (CD32), and/or FcγR3 (CD16). The Fcγ receptors exhibit genetic diversity and, as such, assays for assessing Fcγ receptor binding capacity may employ Fcγ receptors encoded by FCGR genes such as FCGR1A, FCGR2A, FCGR2B, FCGR2C, FCGR3A, and FCGR3B as well as polymorphic variants thereof such as FCGR3AF, FCGR3AH, FCGR3AV, and FCGR3B (NA2).

In some such embodiments, the viral neutralization feature represents the ability of the anti-herpesvirus antibodies in the bodily fluid sample to neutralize a herpesvirus. The Fc viral neutralization feature can be determined using, for example, an in vitro cell-based assay. An exemplary neutralization assay employs human cells (e.g., an epithelial or fibroblast cell line) and a test virus, such as a reporter virus (e.g., a CMV virus strain that expresses a fluorescent protein) to quantify CMV infection in the cells. Neutralizing activity of the antibodies can be evaluated by determining the concentration of the antibody necessary to decrease, for example, 50% of the number of plaques of the test virus.

In some such embodiments, the effector function feature represents the ability of the anti-herpesvirus antibodies in the bodily fluid sample to induce one or more effector functions, such as phagocytosis by monocytes (ADCP) and/or by neutrophils (ADNP), complement deposition (ADCD), antibody dependent cellular cytotoxicity (ADCC).

In some such embodiments, the effector function feature comprises ADCP and/or ADNP. In some such embodiments, the effector function feature comprises ADCD. In some such embodiments, the effector function feature comprises ADCC. In some such embodiments, the effector function feature comprises at least one of ADCP, ADNP, ADCD, and ADCC. In some such embodiments, the effector function feature comprises at least two of ADCP, ADNP, ADCD, and ADCC.

The effector function feature can be determined using, for example, an in vitro cell-based assay. An exemplary ADCC assay employs an FcR-expressing cell (e.g., CD16+ cell) and utilizes one or more readouts, such as target cell lysis or effector cell activation. An exemplary ADCP assay employs monocyte-derived macrophages as the effector cell and evaluates phagocytosis through flow cytometry. Alternatively, effector activation can be measured by a reporter gene such as C1q.

In certain embodiments, any method disclosed herein may comprise the step of providing a therapeutic intervention to a host subject determined to have a latent or primary infection with a herpesvirus (e.g., CMV). In some such embodiments, the step of providing the therapeutic intervention comprises administering a drug to the host subject determined to have a latent or primary infection.

Exemplary drugs include antiviral agents and abortifacients (e.g., if the host subject is pregnant and further determined to be at risk for transmitting the infection to offspring). Exemplary antiviral agents include nucleoside inhibitors such as ganciclovir, valganciclovir, and valacyclovir; DNA terminase complex inhibitors such as letemovir; DNA polymerase inhibitors such as foscarnet; and anti-herpesvirus antibodies such as an anti-herpesvirus monoclonal antibody, polyclonal anti-herpesvirus antibodies, or hyperimmune IVIG (e.g. CMV hyperimmune IVIG). An exemplary antiviral treatment regimen includes intravenously administering 5 mg/kg ganciclovir. Another exemplary antiviral treatment regimen includes orally administering 900 mg valganciclovir once or twice per day. A further exemplary antiviral treatment regimen includes orally or intravenously administering 480 mg letermovir (if letermovir is co-administered with cyclosporine, the dose can be decreased to 240 mg). Exemplary abortifacients include progestin antagonists, such as mifepristone; prostaglandin E1 analogs, such as misoprostol; and antifolates such as methotrexate. Exemplary abortifacient treatment regimens include a combination of mifepristone and misoprostol, such as administering 200 mg mifepristone on day 1 followed 24-48 hours later by 800 μg misoprostol. Misoprostol may be administered buccally, vaginally, or sublingually. In some such embodiments, the step of providing the therapeutic intervention comprises surgical intervention to remove the fetus.

In certain embodiments, any method disclosed herein may comprise the step of monitoring the host subject determined to have a latent or primary infection with a herpesvirus (e.g., CMV) and/or performing one or more secondary clinical tests on such host subject. Exemplary secondary clinical tests include, for example, a blood test, a computed tomography (CT) scan, a magnetic resonance imaging (MRI) scan, an ultrasound scan, an amniocentesis, a non-invasive prenatal test (NIPT), or any combination thereof.

In certain embodiments, any method disclosed herein may comprise the step of referring the host subject determined to have a latent or primary infection with a herpesvirus (e.g., CMV) for further medical examination and/or diagnosis.

In certain embodiments, any method disclosed herein may comprise the step of eliciting an immune response against a herpesvirus in a human subject. In some such embodiments, the human subject has been identified or classified as a suitable candidate for receiving a herpesvirus vaccine.

In some such embodiments, the step of eliciting an immune response against a herpesvirus comprises administering a herpesvirus vaccine to the subject. The herpesvirus vaccine may comprise, for example, live-attenuated virus or glycoprotein antigens or nucleic acids encoding such antigens. For example, the method may comprise administering at least one CMV antigen, or a nucleic acid encoding at least one CMV antigen, to the subject. In some such embodiments the CMV antigen comprises a CMV protein, a CMV protein complex, or antigenic fragment thereof. Exemplary CMV antigens include CMV glycoprotein B (gB) antigens, CMV pentamer complex antigens, and CMV tegument protein antigens. The CMV pentamer complex comprises glycoprotein H (gH), glycoprotein H (gL), glycoprotein UL128, glycoprotein UL130, and glycoprotein UL131A; thus, in some such embodiments, CMV pentamer complex antigens may be derived from such glycoproteins. Exemplary CMV vaccines may comprise a combination of CMV antigens, or a combination of nucleic acids encoding such CMV antigens, such as two, three, four, five, or six CMV antigens. For example, a CMV vaccine may comprise one or all of the following CMV antigens (or a nucleic acid encoding such antigens): gB, gH, gL, UL128, UL130, and UL131A. In some such embodiments, the CMV vaccine is a multivalent vaccine comprising a gB antigen and a pentamer antigen (or nucleic acids encoding such antigens). The gB antigen may comprise the full length of CMV gB or an immunogenic fragment thereof. The gB antigen may comprise one or more amino acid substitutions, additions, and/or deletions relative to wild type CMV gB; such modifications can eliminate a furin cleavage site, prevent the formation of aggregates, and/or improve immunogenicity.

In some such embodiments, a CMV antigen or a nucleic acid encoding such antigen is administered in combination with adjuvant. In some such embodiments, the adjuvant is an immunogenic or immunomodulatory molecule. Exemplary adjuvants include cytokines (e.g., colony stimulating factor (CSF), granulocyte colony stimulating factor (G-CSF), granulocyte-macrophage colony stimulating factor (GM-CSF), and interleukins (IL), such as IL-2, IL-4, IL-7, IL-12, IL-15, IL-21), aluminum hydroxide, and sodium alginate. Exemplary adjuvants also include delivery systems such as lipoplexes (cationic liposomes), which may enhance antigen delivery and/or activate innate immune responses. Further examples of adjuvants include, but are not limited to monophosphoryl-lipid-A (MPL SmithKline Beecham), saponins such as QS21 (SmithKline Beecham), DQS21 (SmithKline Beecham; WO 96/33739), QS7, QS17, QS18, and QS-L1 (So et al., 1997, Mol. Cells 7: 178-186), incomplete Freund's adjuvants, complete Freund's adjuvants, vitamin E, montanid, alum, CpG oligonucleotides (Krieg et al., 1995, Nature 374: 546-549), and various water-in-oil emulsions which are prepared from biologically degradable oils such as squalene and/or tocopherol.

### EXEMPLARY ENUMERATED EMBODIMENTS

Embodiment A1. A method comprising obtaining, by a computing system including one or more computing devices having one or more processors and memory, training data including: first immunological data indicating first immunological features of first subjects having a latent viral infection; and second immunological data indicating second immunological features of second subjects having a primary viral infection; analyzing, by the computing system and using one or more machine learning techniques, the training data to determine a set of immunological features that correspond to latent and/or primary infection and/or time since exposure to and/or infection with a virus; generating, by the computing system, a trained machine learning model that implements the one or more machine learning techniques to determine recency indicators of additional individuals not included in the first subjects or in the second subjects; obtaining, by the computing system, additional immunological data of an additional individual, the additional immunological data indicating values of the set immunological features for the additional individual; and analyzing, by the computing system and using the trained machine learning model, the additional immunological data of the additional individual to determine a recency indicator of the additional individual.

Embodiment A2. The method of embodiment A1, wherein the values of the set of immunological features are included in an input vector that is provided to the trained machine learning model; and the trained machine learning model includes a feature extraction component and a classification component.

Embodiment A3. The method of embodiment A2, wherein the feature extraction component implements a rectified liner unit (ReLU) activation function; and the classification component implements a SoftMax function.

Embodiment A4. The method of embodiment A2 or A3, wherein the feature extraction component comprises a convolutional neural network that includes one or more convolutional layers and one or more max pooling layers; and the classification component includes a number of fully connected layers.

Embodiment A5. The method of embodiment A4, wherein the feature extraction component includes a flattening layer that provides output of the features extraction component as input to the classification component.

Embodiment A6. The method of embodiment A4 or A5, wherein the convolutional neural network includes a first convolutional layer having from 24 filters to 48 filters and a second convolutional layer having from 48 filters to 96 filters.

Embodiment A7. The method of any one of embodiments A1-A6, wherein the recency indicator corresponds to the time since the additional subject was exposed to or infected with a herpesvirus.

Embodiment A8. The method of any one of embodiments A1-A7, comprising performing an assay to obtain the first immunological data, the second immunological data, and the additional immunological data.

Embodiment A9. The method of any one of embodiments A1-A8, wherein the first immunological data, the second immunological data, and the additional immunological data indicate a presence or an absence of a set of antibodies that are produced by subjects in response to the virus.

Embodiment A10. The method of any one of embodiments A1-A8, wherein the set of immunological features correspond to at least one of isotypes of antibodies or subclasses of antibodies present in subjects in which the virus is present.

Embodiment A1. The method of any one of embodiments A1-A10, wherein the set of immunological features corresponds to a measure of glycosylation of antibodies present in subjects in which the virus is present.

Embodiment A12. The method of any one of embodiments A1-A11, wherein the set of immunological features corresponds to a level of effector functions present in subjects in which the virus is present.

Embodiment A13. The method of any one of embodiments A1-A12, wherein the set of immunological features corresponds to at least one of a measure of folding or a measure of unfolding of antigens present in subjects in which the virus is present.

Embodiment A14. The method of any one of embodiments A1-A14, wherein the set of immunological features indicates a specificity of antibodies present in subjects in which the virus is present with respect to at least one of one or more antigens or one or more epitopes of antigens present in the subjects in which the virus is present.

Embodiment B1. A system comprising: one or more hardware processing units; and one or more non-transitory memory devices storing computer-readable instructions that, when executed by the one or more hardware processing units, cause the system to perform operations comprising: obtaining training data including: first immunological data indicating first immunological features of first subjects having a latent viral infection; and second immunological data indicating second immunological features of second subjects having a primary viral infection; analyzing, using one or more machine learning techniques, the training data to determine a set of immunological features that correspond to latent and/or primary infection and/or time since exposure to and/or infection with a virus; generating a trained machine learning model that implements the one or more machine learning techniques to determine recency indicators of additional individuals not included in the first individuals or in the second individuals; obtaining additional immunological data of an additional individual, the additional immunological data indicating values of the set immunological features for the additional individual; and analyzing, using the trained machine learning model, the additional immunological data of the additional individual to determine a recency indicator of the additional individual.

Embodiment B2. The system of embodiment B1, wherein the one or more non-transitory memory devices store additional computer-readable instructions that, when executed by the one or more hardware processing units, cause the system to perform additional operations comprising: performing a training process using the training data to minimize a loss function of the machine learning model.

Embodiment C1. A method for method for eliciting an immune response against a herpesvirus in a human subject, said method comprising:
(a) detecting in a bodily fluid sample from the human subject a set of anti-herpesvirus antibody features;
(b) applying a machine learning algorithm to the anti-herpesvirus antibody features, wherein the machine learning algorithm has importance measures assigned to the anti-herpesvirus antibody features based on data from a plurality of samples obtained from control subjects that had been immunized with a herpesvirus vaccine and wherein the machine learning algorithm assigns an importance measure to one or more anti-herpesvirus antibody features selected from the group consisting of:
   i. isotype,
   ii. subclass,
   iii. Fc receptor binding capacity,
   iv. viral neutralization, and
   v. effector function;
(c) using the machine learning algorithm to classify the human subject as suitable candidate for receiving said herpesvirus vaccine; and
(d) administering said herpesvirus vaccine to the human subject.

1. A computer-implemented system for identifying and/or classifying a subject having a herpesvirus infection or having been exposed to a herpesvirus or antigenic component thereof based on the subject's anti-herpesvirus antibody features, said system comprising a machine learning model trained with data comprising:
(a) viral classifications for subjects, wherein the subjects are classified as having:
(i) a primary herpesvirus infection;
(ii) a latent herpesvirus infection;
(iii) a more recent herpesvirus exposure or infection; and/or
(iv) a less recent herpesvirus exposure or infection;
(b) a set anti-herpesvirus antibody features obtained from said subjects;
wherein the trained machine learning model is configured to analyze a subject's anti-herpesvirus antibody features as input values, and to provide the subject's viral classification as an output value.

2. The system of claim 1, wherein the set of anti-herpesvirus antibody features that includes one or more anti-herpesvirus antibody features selected from the group consisting of:
(i) isotype;
(ii) subclass;
(iii) Fc receptor binding capacity;
(iv) viral neutralization; and
(v) effector function.

3. The system of claim 2, wherein the herpesvirus is cytomegalovirus (CMV) and the set of anti-herpesvirus antibody features is derived from antibodies that specifically recognize a CMV surface or structural protein and/or CMV glycoprotein B (gB), a CMV pentamer complex, or a CMV tegument protein.

4. The system of claim 3, wherein the machine learning model has importance measures assigned to the anti-herpesvirus antibody features.

5. The system of claim 4, wherein the importance measure assigned to the one or more anti-herpesvirus antibody features of subpart (b) is greater than the importance measure assigned to avidity of anti-herpesvirus IgM antibodies and/or avidity of anti-herpesvirus IgG antibodies.

6. A method for treating a subject having a herpesvirus infection, said method comprising:
(a) detecting in a bodily fluid sample from the subject a set of anti-herpesvirus antibody features;
(b) applying a machine learning algorithm to the anti-herpesvirus antibody features, wherein the machine learning algorithm has importance measures assigned to the anti-herpesvirus antibody features based on data from a plurality of samples from individuals having latent and/or primary herpesvirus infection and wherein the machine learning algorithm assigns an importance measure to one or more anti-herpesvirus antibody features selected from the group consisting of:
i. isotype (e.g., including IgA, IgD, IgE, IgG, IgM),
ii. subclass (e.g., including IgA1, IgA2, IgG1, IgG2, IgG3, IgG4),
iii. Fc receptor binding capacity (e.g., including FcγR binding, including FcγR1, FcγR2, FcγR3),
iv. viral neutralization, and
v. effector function (e.g., including phagocytosis by monocytes (ADCP) and/or by neutrophils (ADNP), complement deposition (ADCD), antibody dependent cellular cytotoxicity (ADCC));
(c) using the machine learning algorithm to classify the subject as having a latent or primary herpesvirus infection; and
(d) providing a therapeutic intervention to the subject.

7. The method of claim 6, wherein the herpesvirus is cytomegalovirus (CMV) and the set of anti-herpesvirus antibody features is derived from antibodies that specifically recognize a CMV surface or structural protein and/or CMV glycoprotein B (gB), a CMV pentamer complex, or a CMV tegument protein.

8. The method of claim 6, wherein the importance measure assigned to the one or more anti-herpesvirus antibody features of step (b) is greater than the importance measure assigned to avidity of anti-herpesvirus IgM antibodies and/or avidity of anti-herpesvirus IgG antibodies.

9. The method of claim 6, wherein step (d) comprises administering an antiviral therapy to the subject.

10. The method of claim 9, wherein the antiviral therapy is a monoclonal or polyclonal anti-herpesvirus antibody.

11. A method for treating a subject having a herpesvirus infection, said method comprising:
(a) detecting in a bodily fluid sample from the subject a set of anti-herpesvirus antibody features that includes one or more anti-herpesvirus antibody features selected from the group consisting of:
i. isotype,
ii. subclass,
iii. Fc receptor binding capacity,
iv. viral neutralization, and
v. effector function;
(b) generating an input vector that includes data indicative of the anti-herpesvirus antibody features of the subject;
(c) applying the input vector to a trained machine learning algorithm that is configured to generate an assigned classification to the subject, wherein the assigned classification is one of a plurality of potential classifications of the machine learning algorithm;
(d) determining whether the subject is a suitable candidate for treatment based on the assigned classification; and
(e) responsive to determining that the human subject is suitable, providing a therapeutic intervention to the subject.

12. The method of claim 11, wherein the plurality of potential classifications more recent exposure or infection and less recent exposure or infection.

13. The method of claim 11, wherein the plurality of potential herpesvirus classifications includes primary infection and latent infection.

14. The method of claim 11, wherein the machine learning algorithm is a neural network algorithm.

15. The method of claim 11, wherein the input vector is generated to further include a biophysical profile of the human subject.

16. The method of claim 11, wherein step (e) comprises administering an antiviral therapy to the pregnant subject.

17. A method for treating a subject having a herpesvirus infection, said method comprising: administering an antiviral therapy to the subject, wherein prior to said administration a set of anti-herpesvirus antibody features has been detected in a bodily fluid sample obtained from the subject, wherein said set of anti-herpesvirus antibody features comprises at least one of:
i. isotype,
ii. subclass,
iii. Fc receptor binding capacity,
iv. viral neutralization, and
v. effector function.

18. An in vitro method for identifying a human subject as having latent herpesvirus infection or having herpesvirus primary infection and/or to determining time since exposure or infection, said method comprising:
(a) in a sample which has been obtained from said human subject, measuring one or more anti-herpesvirus antibody features selected from the group consisting of:
i. isotype,
ii. subclass,
iii. Fc receptor binding capacity,
iv. viral neutralization, and
v. effector function;
(b) classifying said subject into a reference cohort selected from a plurality of reference cohorts that have been pre-established by a function of their status as latent or primary herpesvirus infection and/or by time since exposure to or infection with herpesvirus by comparing, by a computer comprising a processing unit, values for the measurements obtained for each feature in step (a) with values, or with a distribution of values, in the plurality of reference cohorts.

19. The method of claim 18, wherein the human subject is a pregnant subject.

20. The method of claim 19, wherein the method is performed prior to parturition.
